(19) United States
(12) Patent Application Publication
(10) Pub. No.: US 2022/0339992 A1
(43) Pub. Date: Oct. 27, 2022

(54) IN-CAR AIR POLLUTION PREVENTION SYSTEM

(71) Applicant: Microjet Technology Co., Ltd., Hsinchu (TW)

(72) Inventors: Hao-Jan Mou, Hsinchu (TW); Chin-Chuan Wu, Hsinchu (TW); Ching-Sung Lin, Hsinchu (TW); Yung-Lung Han, Hsinchu (TW); Chi-Feng Huang, Hsinchu (TW); Chang-Yen Tsai, Hsinchu (TW); Wei-Ming Lee, Hsinchu (TW)

(73) Assignee: Microjet Technology Co., Ltd., Hsinchu (TW)

(21) Appl. No.: 17/677,150

(22) Filed: Feb. 22, 2022

(30) Foreign Application Priority Data
Apr. 23, 2021 (TW) 110114805

(51) Int. Cl. B60H 3/06; A61L 9/16

(52) U.S. Cl. CPC B60H 3/0625; A61L 9/16; B60H 2003/0683; B60H 2003/0691

(57) ABSTRACT

An in-car air pollution prevention system is disclosed and includes plural gas detection modules and a gas conditioning device. The in-car gas detection modules and the out-car gas detection modules are provided for detecting the external gas and the air pollution source and transmitting the gas detection data respectively. A plurality of filtering and purification components are disposed within the gas conditioning device for filtering the external gas and the air pollution source. After the control drive unit compares the gas detection data, the gas conditioning device intelligently select and control the introduction or not introduction of the external gas. The plurality of in-car gas detection modules control the enablement of the gas guider of the gas conditioning device under a monitoring mechanism state. Whereby the gas pollution source in the interior space of the car is filtered through the filtering and purification component, and the gas pollution source is filtered and/or exchanged to generate clean air.

### FIELD OF THE INVENTION

The present disclosure relates to an air pollution filtration executed in a car, and more particularly to an in-car air pollution prevention system.

### BACKGROUND OF THE INVENTION

With the growths of global population and the rapid development in industry, the air quality is deteriorating gradually. It is not only harmful to human health but also life-threatening in severe cases for people to expose in the harmful air pollution gases for a long time.

There are many pollutants in the air, such as carbon dioxide, carbon monoxide, formaldehyde, bacteria, fungi, volatile organic compound (VOC), particulate matter 2.5 (PM_{2.5}) or ozone, etc. which may be seriously harmful to the human body as the concentration of pollutants increases. In the case of PM_{2.5}, such fine particles might penetrate through the alveoli, enter the blood vessels, and circulate throughout the body along with the blood circulation. As a result, they not only might be harmful to the respiratory tract, but also might lead to cardiovascular disease and/or increases the risk of cancer.

Nowadays, the prevalence of epidemic diseases, such as influenza and pneumonia, not only threatens people's health, but also restricts people's social activities, and the willingness to take public transportation has also decreased. As a result, driving by themselves has become the first choice of transportation when people need to go out. Therefore, how to make sure that the gas in the vehicle is clean and safe for people to breath at all times during driving by people becomes an important research and development topic of the present disclosure.

### SUMMARY OF THE INVENTION

An object of the present disclosure is to provide an in-car air pollution prevention system for executing an air pollution filtration in an interior space of a car, so that the air pollution source in the interior space of the car can be filtered rapidly, so as to provide clean, safe and breathable air.

In accordance with an aspect of the present disclosure, an in-car air pollution prevention system is provided and includes a plurality of gas detection modules, a gas conditioning device and a plurality of filtering, and purification components. The plurality of gas detection modules detect the status of gas and the air pollution source, and transmit at least one gas detection datum. The gas conditioning device controls the introduction or not introduction of external gas in an exterior space outside a car into the interior space of the car. The gas conditioning device includes a ventilation channel, a control drive unit and a gas guider, wherein the ventilation channel includes at least one air outlet, at least one air inlet and an external-gas inlet and the gas guider is disposed within the ventilation channel. The control drive unit receives and compares the gas detection data outputted by the gas detection modules. The gas guider guides the discharging of the gas from at least one air outlet and the inhaling of the gas through the external-gas inlet and the at least one air inlet. At least one of the plurality of filtering and purification components is disposed at the at least one air outlet for filtering and purifying the external gas and the air pollution source, at least one of the plurality of filtering and purification components is disposed at the at least one air inlet for filtering and purifying the air pollution source, and at least one of the plurality of gas detection modules is disposed at two sides of the plurality of filtering and purification components, respectively. After the control drive unit compares the gas detection data, the gas conditioning device is allowed to intelligently select and control the introduction or not introduction of the external gas in the exterior space into the interior space, and the gas guider of the gas conditioning device is instantly controlled to be enabled by the plurality of gas detection modules in a state of a monitoring mechanism for filtering and purifying the air pollution source in the interior space through the plurality of filtering and purification components, so that the air pollution source in the interior space is filtered and exchanged to provide clean air.

### BRIEF DESCRIPTION OF THE DRAWINGS

The above contents of the present disclosure will become more readily apparent to those ordinarily skilled in the art after reviewing the following detailed description and accompanying drawings, in which:

FIG 1A is an exemplary schematic diagram illustrating a gas detection module and a gas conditioning device installed in a car according to the embodiment of the present disclosure;

FIG. 1B is an exemplary schematic diagram illustrating the gas conditioning device installed in the car according to the embodiment of the present disclosure;

FIG. 2A is a schematic cross-sectional view illustrating the gas conditioning device and the filtering and purification component according to the embodiment of the present disclosure;

FIG. 2B is an exemplary schematic diagram illustrating the gas detection modules, the gas conditioning device and the filtering and purification components installed in the car according to the embodiment of the present disclosure;

FIG. 2C is a schematic cross-sectional view illustrating the air outlet of the gas conditioning device and the filtering and purification component according to the embodiment of the present disclosure;

FIG. 2D is a schematic cross-sectional view illustrating the air inlet of the gas conditioning device and the filtering and purification component according to the embodiment of the present disclosure;

FIG. 2E is a schematic cross-sectional view illustrating the filtering and purification component according to the embodiment of the present disclosure;

FIG. 3 is a schematic perspective view illustrating the gas detection module according to the embodiment of the present disclosure;

FIG. 4A is a schematic front perspective view illustrating the gas detection main part according to the embodiment of the present disclosure;

FIG. 4B is a schematic rear perspective view illustrating the gas detection main part according to the embodiment of the present disclosure;

FIG. 4C is an exploded view illustrating the gas detection main part according to the embodiment of the present disclosure;

FIG. 5A is a schematic front perspective view illustrating the base according to the embodiment of the present disclosure;

FIG. 5B is a schematic rear perspective view illustrating the base according to the embodiment of the present disclosure;

FIG. 6 is a schematic view illustrating the laser component combined within the base according to the embodiment of the present disclosure;

FIG. 7A is a schematic exploded view illustrating the combination of the piezoelectric actuator and the base according to the embodiment of the present disclosure;

FIG. 7B is a schematic perspective view illustrating the combination of the piezoelectric actuator and the base according to the embodiment of the present disclosure;

FIG. 8A is a schematic exploded front view illustrating the piezoelectric actuator according to the embodiment of the present disclosure;

FIG. 8B is a schematic exploded rear view illustrating the piezoelectric actuator according to the embodiment of the present disclosure;

FIG. 9A is a schematic cross-sectional view illustrating the piezoelectric actuator according to the embodiment of the present disclosure;

FIG. 9B is a schematic cross-sectional view illustrating a first operation step of the piezoelectric actuator according to the embodiment of the present disclosure;

FIG. 9C is a schematic cross-sectional view illustrating a second operation step of the piezoelectric actuator according to the embodiment of the present disclosure;

FIG. 10A is a schematic cross-sectional view illustrating the gas of the gas detection main part entering through the inlet opening of the outer cover according to the embodiment of the present disclosure;

FIG. 10B is a schematic cross-sectional view illustrating the light beam emitted by the laser component emitting a light beam to pass through the transparent window and be irradiated into the gas-inlet groove according to the embodiment of the present disclosure;

FIG. 10C is a schematic cross-sectional view illustrating the gas in the gas-outlet groove being pushed to flow through the gas outlet and the outlet opening and discharged out according to the embodiment of the present disclosure;

FIG. 11 is a schematic diagram illustrating a telecommunication connecting configuration of the in-car gas detection module, the out-car gas detection module and the control drive unit according to the embodiment of the present disclosure; and

FIG. 12 is a block diagram illustrating a telecommunication connecting configuration of the in-car gas detection module, the out-car detection module, the controlling circuit board and the purification and filtration device according to the embodiment of the present disclosure.

### DETAILED DESCRIPTION OF THE PREFERRED EMBODIMENT

The present disclosure will now be described more specifically with reference to the following embodiments. It is to be noted that the following descriptions of preferred embodiments of this invention are presented herein for purpose of illustration and description only. It is not intended to be exhaustive or to be limited to the precise form disclosed.

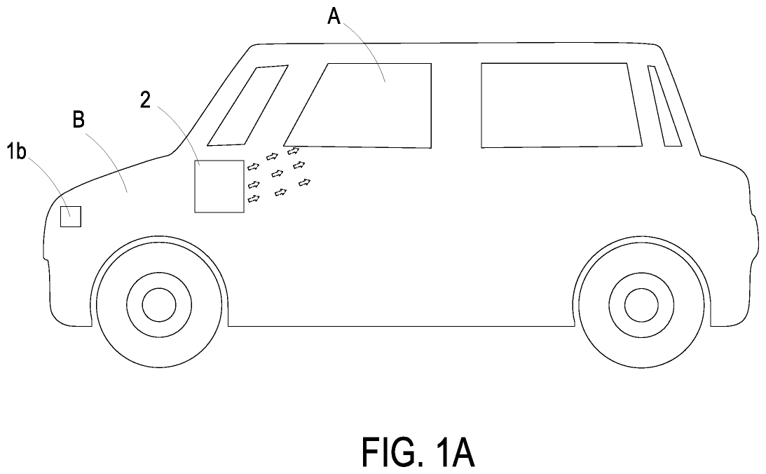

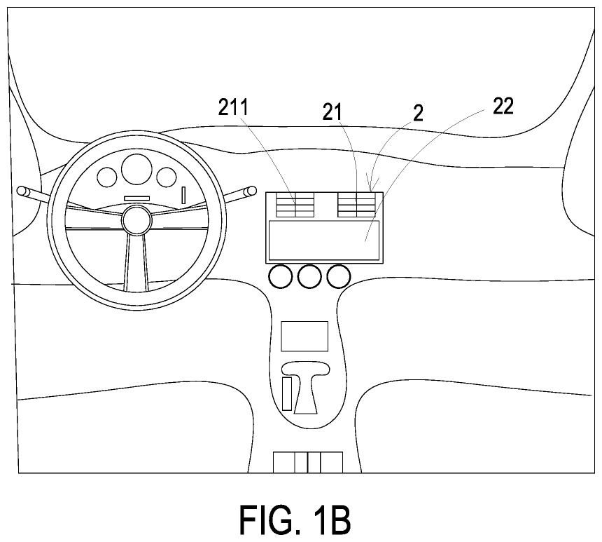

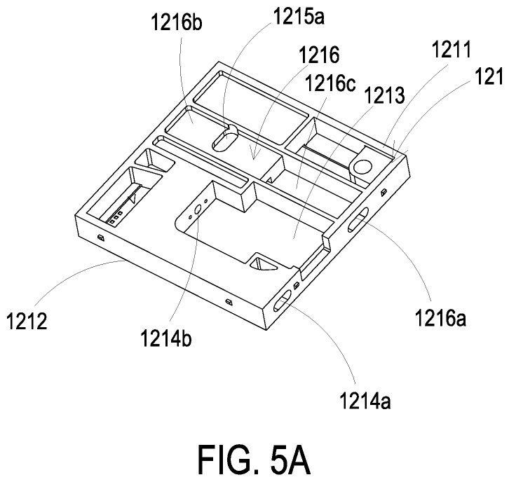

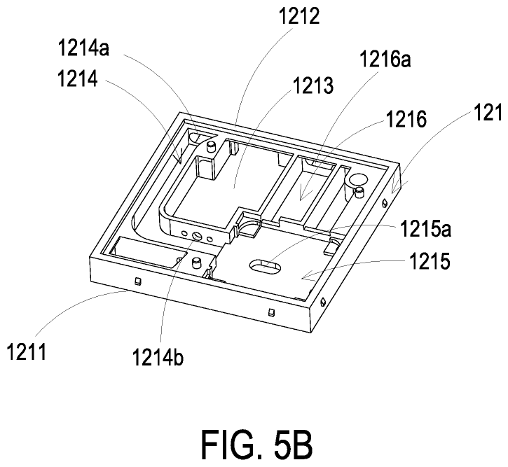

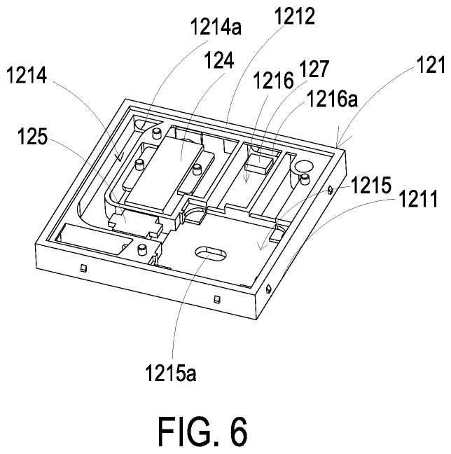

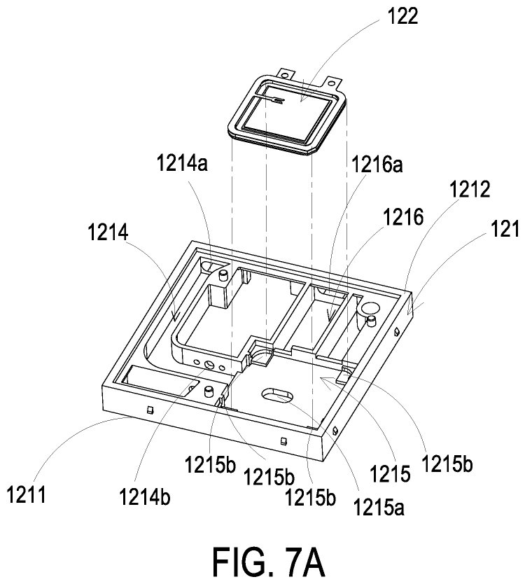

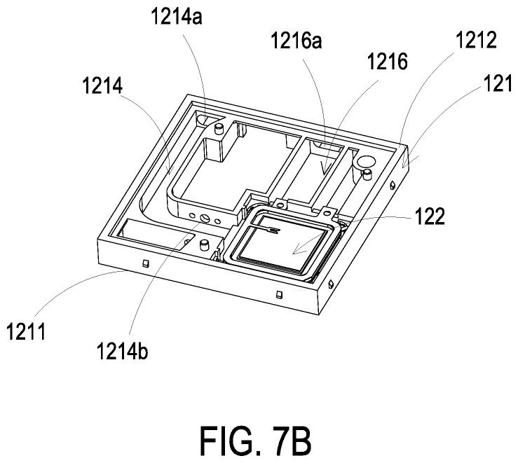

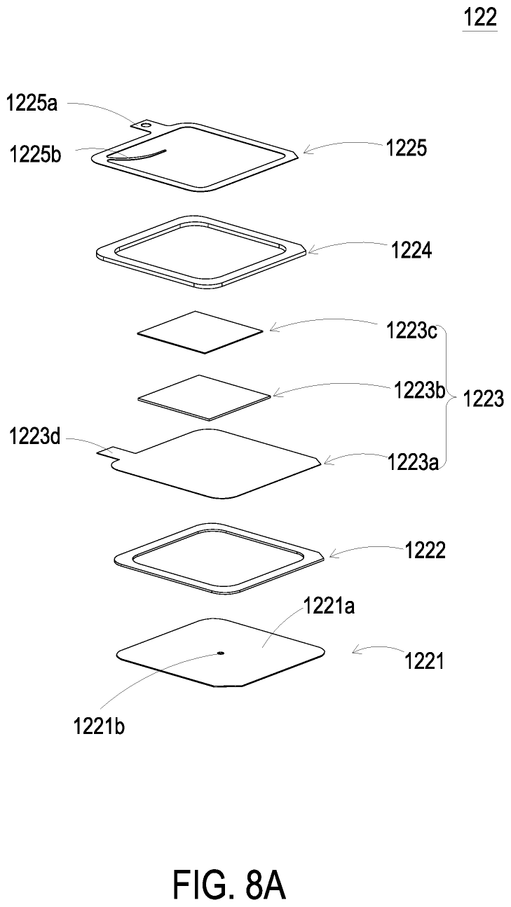

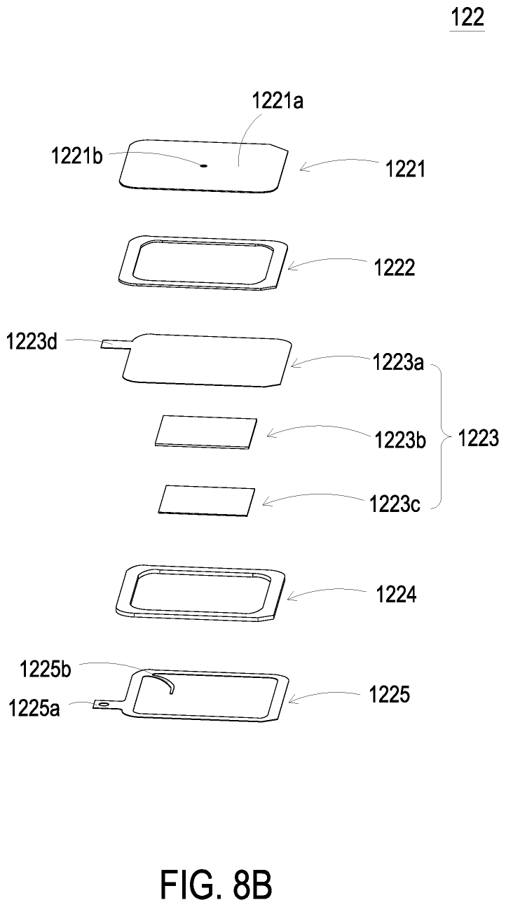

Please refer to FIGS. 1 to 12. The present disclosure provides an in-car air pollution prevention system for exchanging and filtering air pollution source in an interior space A of a car, so that the air pollution source in the interior space A of the car is filtered rapidly, so as to provide clean, safe and breathable air. The in-car air pollution prevention system includes a plurality of gas detection modules, a gas conditioning device 2 and a plurality of filtering and purification components D.

In the embodiment, the plurality of gas detection modules includes a plurality of in-car gas detection modules 1a and a plurality of out-car gas detection modules 1b. The in-car gas detection module 1a is disposed at the position of an external-gas inlet 213 in the interior space A of the car for detecting an air pollution source in the interior space A of the car and transmitting an in-car gas detection datum. Preferably but not exclusively, in the embodiment, the in-car gas detection module 1a is a mobile detection device. That is, the in-car gas detection module 1a may be a wearable device, such as a watch or a bracelet, which is directly worn on the human body (not shown). When people get into the interior space A of the car, the in-car gas detection module 1b can detect the air pollution source in the interior space A of the car immediately in real-time at any time. In the embodiment, the out-car gas detection module 1b is disposed in an exterior space B outside the car for detecting the external gas in the exterior space B outside the car and transmitting an out-car gas detection datum.

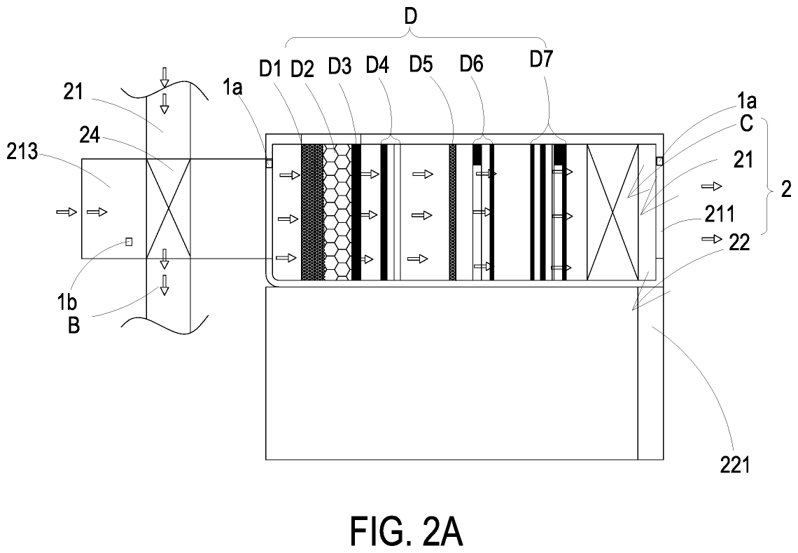

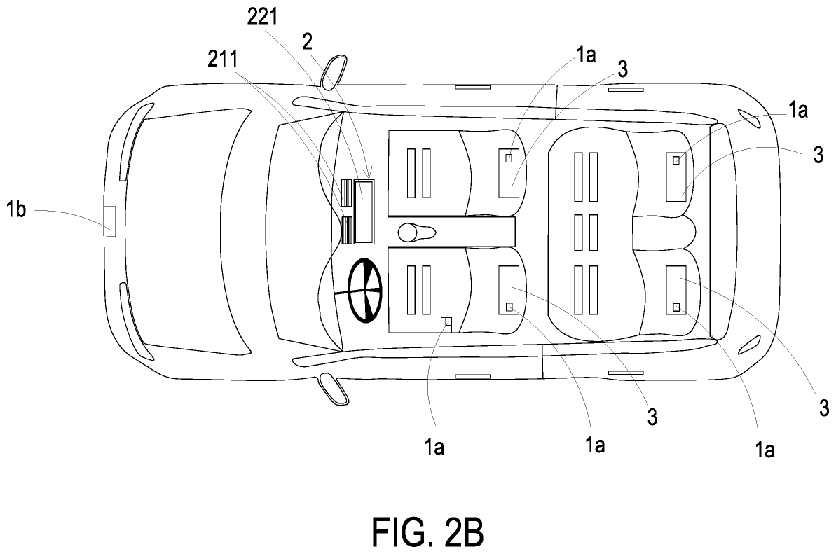

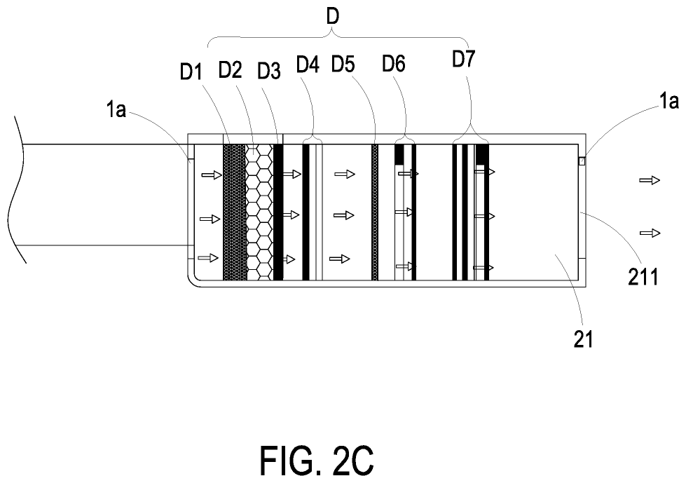

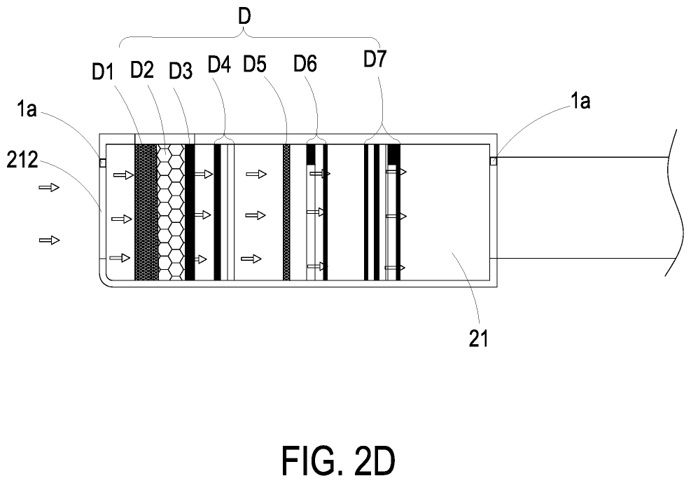

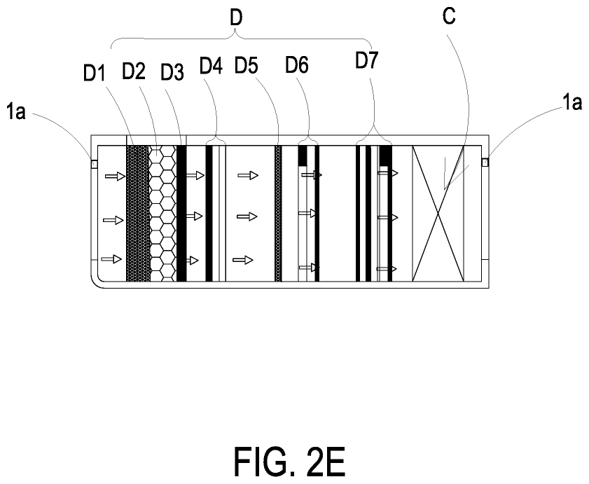

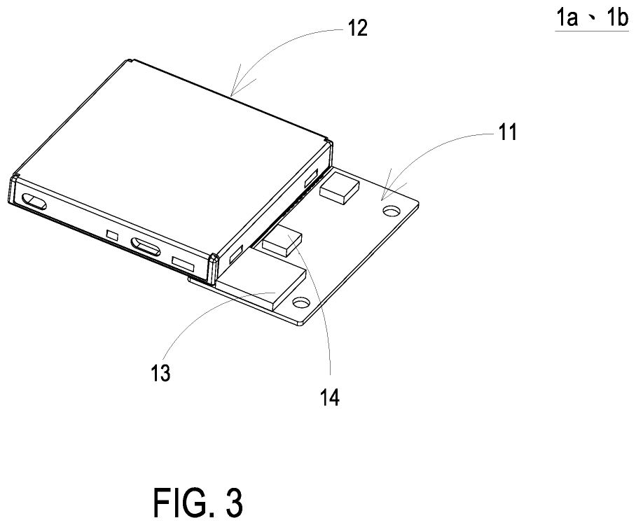

In the embodiment, the gas conditioning device 2 controls the introduction or not introduction of the external gas in an exterior space B outside the car into the interior space A of the car. In addition, the gas conditioning device 2 includes a ventilation channel 21, a control drive unit 22 and an intake-control element 24. The control drive unit 22 includes a touch screen 221 for setting up the control instructions of the gas conditioning device 2 by touching the touch screen 221 and displaying the in-car gas detection datum. As shown in FIGS. 2A to 2D, the ventilation channel 21 includes at least one air outlet 211, at least one air inlet 212 and an external-gas inlet 213, and a gas guider C is disposed therein. Preferably but not exclusively, the gas guider C guides the air to be discharge from the at least one air outlet 211 to, and inhale through the external-gas inlet 213 and the at least one air inlet 212. As shown in FIG. 2C and FIG. 3D, the at least one air outlet 211 is in fluid communication with the ventilation channel 21 shown in FIG. 2A. In that, after the gas is discharged out through the air outlet 211, the discharged gas flows through the ventilation channel 21 and transported to the exterior space B outside the car.

In the embodiment, the filtering and purification components D are disposed at the positions of the air outlet 211 and the air inlet 212 of the ventilation channel 21 for filtering and purifying the external gas and the air pollution source. Moreover, two in-car gas detection modules 1a are disposed at two sides of the filtering and purification component D, respectively. Based on the gas detection data before and after filtering are detected by the in-car gas detection modules 1a on both sides, the control drive unit 22 is allowed to receive and compare the out-car gas detection datum outputted by the out-car gas detection module 1b and the in-car gas detection datum outputted by the in-car gas detection module 1a, so that the filter and purification component D is driven to filter the external gas and the air pollution source, so as to generate and introduce clean air into the interior space A of the car.

In the embodiment, the control drive unit 22 of the gas conditioning device 2 controls the opening and closing of the air intake control member 24, and receives and compares the in-car gas detection datum outputted from the in-car gas detection module 1a and the out-car gas detection datum outputted from the out-car gas detection module 1b, so as to determine the opening and closing of the external-gas inlet 213 and selectively control the introduction or not introduction of the external gas in the exterior space B outside the car into the interior space A of the car, and control the gas guider C of the gas conditioning device 2 to be enabled immediately in a state of a monitoring mechanism. Please refer to FIG. 2A and FIG. 2D. The external-gas inlet 213 and the air inlet 212 are controlled by the intake-control element 24 to open for introducing the external gas in the exterior space B outside the car and the air pollution source in the interior space A of the car, respectively, into the gas conditioning device 2. The external gas and the air pollution source are filtered through the filtering and purification components D and exhausted into the exterior space B outside the car through the ventilation channel 21.

Preferably but not exclusively, in an embodiment, the control drive unit 22 of the gas conditioning device 2 is configured to receive and compare the in-car gas detection data outputted by at least three in-car gas detection modules 1a under the calculation of artificial intelligence, so as to locate the position of the air pollution source in the interior space A of the car, and intelligently select and control the filtering and purification component D disposed in the position of the air inlet 212 adjacent to the air pollution source to accelerate the transportation for filtering.

Preferably but not exclusively, in another embodiment, the control drive unit 22 of the gas conditioning device 2 is configured to receive and compare the in-car gas detection data outputted by at least three in-car gas detection modules 1a under the calculation of artificial intelligence, so as to locate the position of the air pollution source in the interior space A of the car, and intelligently select and control the air outlet 211 adjacent to the air pollution source to expel gas in first priority, and direct the air pollution source toward the air inlet 212 adjacent thereto. At the same time, the control drive unit 22 of the gas conditioning device 2 selects and controls the rest of the air outlets 211 to expel gas under the calculation of artificial intelligence, so as to generate an airflow to direct the air pollution source toward the air inlet 212 adjacent to the air pollution source and to be inhaled for filtering rapidly.

In the embodiment, the state of the monitoring mechanism is enabled when at least one of the gas detection data of the air pollution source detected by the plurality of gas detection modules in the interior space A of the car is greater than a safe detection value. Preferably but not exclusively, the safe detection value comprises at least one selected from the group consisting of PM2.5 less than 35 μg/m³, carbon dioxide content less than 1000 ppm, total volatile organic compounds (TVOC) less than 0.56 ppm, formaldehyde content less than 0.08 ppm, the amount of bacteria less than 1500 CFU/m³, the amount of fungi less than 1000 CFU/m³, sulfur dioxide content less than 0.075 ppm, nitrogen dioxide content less than 0.1 ppm, carbon monoxide content less than 9 ppm, ozone content less than 0.06 ppm, lead content less than 0.15 μg/m³ and a combination thereof.

Please refer to FIGS. 2A and 2B. In another embodiment, the in-car air pollution prevention system of the present disclosure further includes at least one purification and filtration device 3. The purification and filtration device 3 includes the gas guider C and the filtering and purification component D. The in-car gas detection module 1a is combined with the purification and filtration device 3. In the embodiment, the in-car gas detection module 1a transmits the in-car gas detection datum to the control drive unit 22 of the gas conditioning device 2, and the control drive unit 22 of the gas conditioning device 2 receives and compares the in-car gas detection datum, so as to intelligently select and control the enabling of purification and filtration device 3 adjacent to the air pollution source. In that, the gas guider C is enabled, and the air pollution source in the interior space A of the car is guided and enters the filtering and purification component D of the purification and filtration device 3 for filtering and purifying. Preferably but not exclusively, in one embodiment, the purification and filtration device 3 is combined and embedded on a trim panel, a seat or a door pillar in the interior space A of the car.

Preferably but not exclusively, in an embodiment, the control drive unit 22 of the gas conditioning device 2 is configured to receive and compare the in-car gas detection data outputted by at least three in-car gas detection modules 1a under the calculation of artificial intelligence, so as to locate the position of the air pollution source in the interior space A of the car, and intelligently select and control the enabling of the purification and filtration device 3 adjacent to the air pollution source, so the the air pollution source can be inhaled into the purification and filtration device 3 without diffusion to accelerate the filtration.

Preferably but not exclusively, in another embodiment, the control drive unit 22 of the gas conditioning device 2 is configured to receive and compare the in-car gas detection data outputted by at least three in-car gas detection modules 1a under the calculation of artificial intelligence, so as to locate the position of the air pollution source in the interior space A of the car, and intelligently select and control the purification and filtration device 3 adjacent to the air pollution source to be enabled in first priority. At the same time, the control drive unit 22 of the gas conditioning device 2 selects and controls the rest of the purification and filtration devices 3 to be enabled under the calculation of artificial intelligence, so that an airflow is generated to direct the air pollution source toward the purification and filtration device 3 adjacent to the air pollution source to be inhaled for filtering rapidly.

In the embodiment, at least one of the plurality of in-car gas detection modules 1a is disposed at two sides of the plurality of purification and filtration devices 3, respectively. Based on the in-car gas detection data before and after filtering are detected by the in-car gas detection modules 1a on both sides, the control drive unit 22 can receive and compare the in-car gas detection data outputted by the in-car gas detection modules 1a located at the positions of the plurality of the purification and filtration devices 3 to ensure the air pollution source is filtered by the plurality of the purification and filtration devices 3, so as to generate another clean air introduced into the interior space A of the car.

In the embodiment, the state of the monitoring mechanism is enabled when at least one of the gas detection data of the air pollution source detected by the plurality of gas detection modules in the interior space A of the car is greater than a safe detection value. Preferably but not exclusively, the safe detection value comprises at least one selected from the group consisting of PM2.5 less than 35 μg/m³, carbon dioxide content less than 1000 ppm, total volatile organic compounds (TVOC) less than 0.56 ppm, formaldehyde content less than 0.08 ppm, the amount of bacteria less than 1500 CFU/m³, the amount of fungi less than 1000 CFU/m³, sulfur dioxide content less than 0.075 ppm, nitrogen dioxide content less than 0.1 ppm, carbon monoxide content less than 9 ppm, ozone content less than 0.06 ppm, lead content less than 0.15 μg/m³ and a combination thereof.

In the embodiment, the filtering and purification component D includes a combination of various implementations. Preferably but not exclusively, the filtering and purification component D can be a combination of an activated carbon D1 and a high efficiency particulate air (HEPA) filter screen D2; or a combination of an activated carbon D1, a high efficiency particulate air (HEPA) filter screen D2 and a zeolite screen D3. The activated carbon D1 is configured to filter and absorb the particulate matter 2.5 (PM_{2.5}), the zeolite screen D3 is configured to filter and absorb the volatile organic compounds (VOC), and the HEPA filter screen D2 is configured to absorb the chemical smoke, bacteria, dust particles and pollen contained in the gas, so that the air pollution source introduced into the filtering and purification component D is filtered and purified to achieve the effect of filtering and purifying. In some embodiment, the HEPA filter screen D2 is coated with a cleansing factor containing chlorine dioxide layer, so as to inhibit viruses, bacteria and fungi contained in gas introduced into the filtering and purification component D. Preferably but not exclusively, the HEPA filter screen D2 is coated with a cleansing factor containing chlorine dioxide layer, so as to inhibit viruses, bacteria, fungi, influenza A, influenza B, enterovirus and norovirus in the air pollution source introduced into the filtering and purification component D. The inhibition ratio is more than 99%, and it is helpful of reducing the cross-infection of viruses. In some embodiment, the HEPA filter screen D2 is coated with an herbal protective layer extracted from ginkgo and Japanese rhus chinensis to form an herbal protective anti-allergic filter, so as to resist allergy effectively and destroy a surface protein of influenza virus (H1N1) introduced by the filtering and purification component D and passing through the HEPA filter screen D2. In some embodiment, the HEPA filter screen D2 is coated with a silver ion, so as to inhibit viruses and bacteria contained in the air pollution source introduced by the filtering and purification component D.

In an embodiment, the filtering and purification component D includes the combination of an activated carbon D1, a high efficiency particulate air (HEPA) filter screen D2 and a zeolite screen D3, and a phot-catalyst unit D4. In that, the air pollution source is introduced into the filtering and purification component D and the light energy is converted into the chemical energy by the photo-catalyst unit D4, thereby decomposing harmful material in the air pollution source and disinfecting bacteria contained therein, so as to achieve the effects of filtering and purifying.

In an embodiment, the filtering and purification component D includes the combination of an activated carbon D1, a high efficiency particulate air (HEPA) filter screen D2 and a zeolite screen D3, and a photo-plasma unit D5. The photo-plasma unit D5 includes a nanometer irradiation tube. The air pollution source introduced by the filtering and purification component D is irradiated by the nanometer irradiation tube to decompose and purify volatile organic compounds contained in the air pollution source. When the air pollution source is introduced by the filtering and purification component D, the introduced gas is irradiated by the nanometer irradiation tube, thereby oxygen molecules and water molecules contained in the air pollution are decomposed into high oxidizing photo-plasma, and generates an ion flow capable of destroying organic molecules. In that, volatile formaldehyde, volatile toluene and volatile organic compounds (VOC) contained in the air pollution are decomposed into water and carbon dioxide, so as to achieve the effects of filtering and purifying.

In an embodiment, the filtering and purification component D includes the combination of an activated carbon D1, a high efficiency particulate air (HEPA) filter screen D2 and a zeolite screen D3, and a negative ionizer D6. When the air pollution source introduced by the filtering and purification component D passes through a high voltage discharge, it makes the suspended particles in the air pollution source to carry with positive charge and adhered to the dust collecting plate carry with negative charges, so as to achieve the effects of filtering and purifying the air pollution source introduced.

In an embodiment, the filtering and purification component D includes the combination of an activated carbon D1, a high efficiency particulate air (HEPA) filter screen D2 and a zeolite screen D3, and a plasma ion unit D7. A high-voltage plasma column with plasma ion is formed by the plasma ion unit D7, so as to decompose viruses or bacteria contained in the air pollution source introduced by the filtering and purification component D. The oxygen molecules and water molecules contained in the air pollution source are decomposed into positive hydrogen ions (H⁺) and negative oxygen ions (O₂⁻) by the plasma ion. The substances attached with water around the ions are adhered on the surface of viruses and bacteria and converted into OH radicals with extremely strong oxidizing power, thereby removing hydrogen (H) from the protein on the surface of viruses and bacteria, and thus decomposing (oxidizing) the protein, so as to filter the introduced air pollution source and achieve the effects of filtering and purifying.

In an embodiment, the filtering and cleaning component D may merely include the HEPA filter screen D2. In an embodiment, the HEPA filter screen D2 is combined with any one of the phot-catalyst unit D4, the photo-plasma unit D5, the negative ionizer D6 and the plasma ion unit D7. In an embodiment, the HEPA filter screen D2 is combined with a combination of any two of the phot-catalyst unit D4, the photo-plasma unit D5, the negative ionizer D6 and the plasma ion unit D7. In an embodiment, the HEPA filter screen D2 is combined with a combination of any three of the phot-catalyst unit D4, the photo-plasma unit D5, the negative ionizer D6 and the plasma ion unit D7. Alternatively, the HEPA filter screen D2 is combined with the phot-catalyst unit D4, the photo-plasma unit D5, the negative ionizer D6 and the plasma ion unit D7.

Notably, the service life of the HEPA filter screen D2 is calculated based on the monitoring mechanism of the gas detection data detected by the in-car gas detection module 1a and the out-car gas detection module 1a with reference to an accumulated operation time of the gas guider C in the gas conditioning device 2.

After understanding the method of notifying in-car air pollution of the present disclosure, the implementation devices for the method of notifying in-car air pollution of the present disclosure are described in detail as following.

Please refer to FIG. 3. In the embodiment, each of the in-car gas detection module 1a and the out-car gas detection module 1b includes a controlling circuit board 11, a gas detection main part 12, a microprocessor 13 and a communicator 14. The gas detection main part 12, the microprocessor 13 and the communicator 14 are integrally packaged on the controlling circuit board 11 and electrically connected to each other. In the embodiment, the microprocessor 13 and the communicator 14 are mounted on the controlling circuit board 11. The microprocessor 13 controls the driving signal of the gas detection main part 12 for enabling the detection, and receives the detection information of the air pollution source detected by the gas detection main part 12 for calculating, processing and outputting, and the communicator is used for external communication transmission. The detection information of the gas detection main part 12 is converted into the detection datum for storage. The communicator 14 receives the gas detection datum output by the microprocessor 13, and transmits the gas detection datum to a cloud processing device (not shown) or an external device (not shown). Preferably but not exclusively, the external device is a portable mobile device (not shown). The above-mentioned external communication transmission of the communicator 14 can be a wired two-way communication transmission or a wireless two-way communication transmission. Preferably but not exclusively, the wired two-way communication transmission is one selected form the group consisting of a USB communication transmission, a mini-USB communication transmission and a micro-USB communication transmission. Preferably but not exclusively, the wireless two-way communication transmission is one selected from the group consisting of a Wi-Fi communication transmission, a Bluetooth communication transmission, a radio frequency identification communication transmission and a near field communication (NFC) transmission.

In the embodiment, the air pollution source is at least one selected from the group consisting of particulate matter, carbon monoxide, carbon dioxide, ozone, sulfur dioxide, nitrogen dioxide, lead, total volatile organic compounds (TVOC), formaldehyde, bacteria, fungi, virus and a combination thereof.

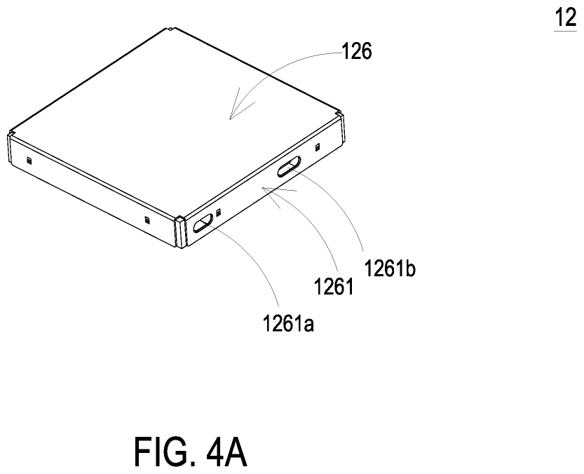

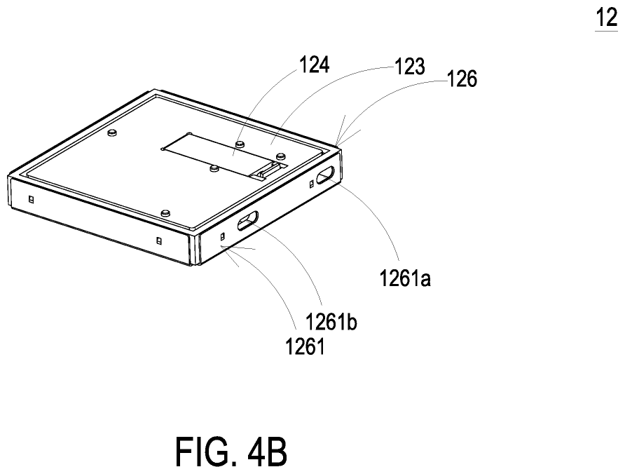

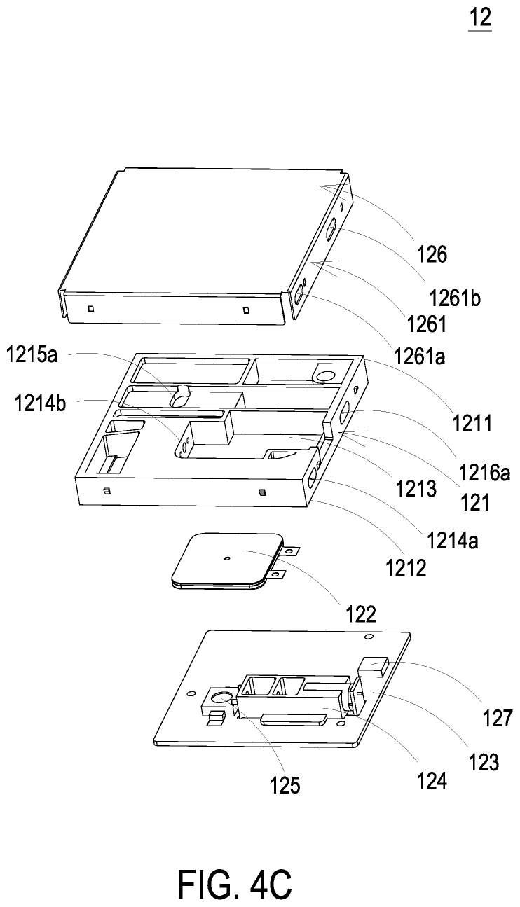

Please refer to FIG. 4A to FIG. 9A. In the embodiment, the gas detection main part 12 includes a base 121, a piezoelectric actuator 122, a driving circuit board 123, a laser component 124, a particulate sensor 125, an outer cover 126 and a gas sensor 127. In the embodiment, the base 121 includes a first surface 1211, a second surface 1212, a laser loading region 1213, a gas-inlet groove 1214, a gas-guiding-component loading region 1215 and a gas-outlet groove 1216. The first surface 1211 and the second surface 1212 are two surfaces opposite to each other. In the embodiment, the laser loading region 1213 is hollowed out from the first surface 1211 toward the second surface 1212. The outer cover 126 covers the base 121 and includes a side plate 1261. The side plate 1261 has an inlet opening 1261a and an outlet opening 1261b. The gas-inlet groove 1214 is concavely formed from the second surface 1212 and disposed adjacent to the laser loading region 1213. The gas-inlet groove 1214 includes a gas-inlet 1214a and two lateral walls. The gas-inlet 1214a is in communication with an environment outside the base 121, and is spatially corresponding in position to an inlet opening 1261a of the outer cover 126. Two transparent windows 1214b are opened on the two lateral walls of the gas-inlet groove 1214 and are in communication with the laser loading region 1213. Therefore, the first surface 1211 of the base 121 is covered and attached by the outer cover 126, and the second surface 1212 is covered and attached by the driving circuit board 123, so that an inlet path is defined by the gas-inlet groove 1214.

In the embodiment, the gas-guiding-component loading region 1215 mentioned above is concavely formed from the second surface 1212 and in communication with the gas-inlet groove 1214. A ventilation hole 1215a penetrates a bottom surface of the gas-guiding-component loading region 1215. The gas-guiding-component loading region 1215 includes four positioning protrusions 1215b disposed at four corners of the gas-guiding-component loading region 1215, respectively. In the embodiment, the gas-outlet groove 1216 includes a gas-outlet 1216a, and the gas-outlet 1216a is spatially corresponding to the outlet opening 1261b of the outer cover 126. The gas-outlet groove 1216 includes a first section 1216b and a second section 1216c. The first section 1216b is concavely formed out from the first surface 1211 in a region spatially corresponding to a vertical projection area of the gas-guiding-component loading region 1215. The second section 1216c is hollowed out from the first surface 1211 to the second surface 1212 in a region where the first surface 1211 is extended from the vertical projection area of the gas-guiding-component loading region 1215. The first section 1216b and the second section 1216c are connected to form a stepped structure. Moreover, the first section 1216b of the gas-outlet groove 1216 is in communication with the ventilation hole 1215a of the gas-guiding-component loading region 1215, and the second section 1216c of the gas-outlet groove 1216 is in communication with the gas-outlet 1216a. In that, when first surface 1211 of the base 121 is attached and covered by the outer cover 126 and the second surface 1212 of the base 121 is attached and covered by the driving circuit board 123, the gas-outlet groove 1216 and the driving circuit board 123 collaboratively define an outlet path.

In the embodiment, the laser component 124 and the particulate sensor 125 are disposed on and electrically connected to the driving circuit board 123 and located within the base 121. In order to clearly describe and illustrate the positions of the laser component 124 and the particulate sensor 125 in the base 121, the driving circuit board 123 is intentionally omitted. The laser component 124 is accommodated in the laser loading region 1213 of the base 121, and the particulate sensor 125 is accommodated in the gas-inlet groove 1214 of the base 121 and is aligned to the laser component 124. In addition, the laser component 124 is spatially corresponding to the transparent window 1214b, therefore, a light beam emitted by the laser component 124 passes through the transparent window 1214b and is irradiated into the gas-inlet groove 1214. A light beam path emitted from the laser component 124 passes through the transparent window 1214b and extends in an orthogonal direction perpendicular to the gas-inlet groove 1214. In the embodiment, a projecting light beam emitted from the laser component 124 passes through the transparent window 1214b and enters the gas-inlet groove 1214 to irradiate the suspended particles contained in the gas passing through the gas-inlet groove 1214. When the suspended particles contained in the gas are irradiated and generate scattered light spots, the scattered light spots are received and calculated by the particulate sensor 125 to obtain the gas detection information. In the embodiment, the gas sensor 127 is positioned and disposed on the driving circuit board 123, electrically connected to the driving circuit board 123, and accommodated in the gas-outlet groove 1216, so as to detect the air pollution source introduced into the gas-outlet groove 1216. Preferably but not exclusively, in an embodiment, the gas sensor 127 includes a volatile-organic-compound sensor for detecting the information of carbon dioxide (CO₂) or volatile organic compounds (TVOC). Preferably but not exclusively, in an embodiment, the gas sensor 127 includes a formaldehyde sensor for detecting the information of formaldehyde (HCHO) gas. Preferably but not exclusively, in an embodiment, the gas sensor 127 includes a bacteria sensor for detecting the information of bacteria or fungi. Preferably but not exclusively, in an embodiment, the gas sensor 127 includes a virus sensor for detecting the information of virus in the gas. Preferably but not exclusively, the gas sensor 127 is a temperature and humidity sensor for detecting the temperature and humidity information of the gas.

In the embodiment, the piezoelectric actuator 122 is accommodated in the square-shaped gas-guiding-component loading region 1215 of the base 121. In addition, the gas-guiding-component loading region 1215 of the base 121 is in fluid communication with the gas-inlet groove 1214. When the piezoelectric actuator 122 is enabled, the gas in the gas-inlet groove 1214 is inhaled by the piezoelectric actuator 122, so that the gas flows into the piezoelectric actuator 122, and is transported into the gas-outlet groove 1216 through the ventilation hole 1215a of the gas-guiding-component loading region 1215. Moreover, the driving circuit board 123 covers the second surface 1212 of the base 121, and the laser component 124 is positioned and disposed on the driving circuit board 123, and is electrically connected to the driving circuit board 123. The particulate sensor 125 is also positioned and disposed on the driving circuit board 123 and electrically connected to the driving circuit board 123. In that, when the outer cover 126 covers the base 121, the inlet opening 1261a is spatially corresponding to the gas-inlet 1214a of the base 121, and the outlet opening 126 lb is spatially corresponding to the gas-outlet 1216a of the base 121.

In the embodiment, the piezoelectric actuator 122 includes a gas-injection plate 1221, a chamber frame 1222, an actuator element 1223, an insulation frame 1224 and a conductive frame 1225. In the embodiment, the gas-injection plate 1221 is made by a flexible material and includes a suspension plate 1221a and a hollow aperture 1221b. The suspension plate 1221a is a sheet structure and is permitted to undergo a bending deformation. Preferably but not exclusively, the shape and the size of the suspension plate 1221a are accommodated in the inner edge of the gas-guiding-component loading region 1215, but not limited thereto. The hollow aperture 1221b passes through a center of the suspension plate 1221a, so as to allow the gas to flow therethrough. Preferably but not exclusively, in the embodiment, the shape of the suspension plate 1221a is selected from the group consisting of a square, a circle, an ellipse, a triangle and a polygon, but not limited thereto.

In the embodiment, the chamber frame 1222 is carried and stacked on the gas-injection plate 1221. In addition, the shape of the chamber frame 1222 is corresponding to the gas-injection plate 1221. The actuator element 1223 is carried and stacked on the chamber frame 1222. A resonance chamber 1226 is collaboratively defined by the actuator element 1223, the chamber frame 1222 and the suspension plate 1221a and is formed between the actuator element 1223, the chamber frame 1222 and the suspension plate 1221a. The insulation frame 1224 is carried and stacked on the actuator element 1223 and the appearance of the insulation frame 1224 is similar to that of the chamber frame 1222. The conductive frame 1225 is carried and stacked on the insulation frame 1224, and the appearance of the conductive frame 1225 is similar to that of the insulation frame 1224. In addition, the conductive frame 1225 includes a conducting pin 1225a and a conducting electrode 1225b. The conducting pin 1225a is extended outwardly from an outer edge of the conductive frame 1225, and the conducting electrode 1225b is extended inwardly from an inner edge of the conductive frame 1225.

Moreover, the actuator element 1223 further includes a piezoelectric carrying plate 1223a, an adjusting resonance plate 1223b and a piezoelectric plate 1223c. The piezoelectric carrying plate 1223a is carried and stacked on the chamber frame 1222. The adjusting resonance plate 1223b is carried and stacked on the piezoelectric carrying plate 1223a. The piezoelectric plate 1223c is carried and stacked on the adjusting resonance plate 1223b. The adjusting resonance plate 1223b and the piezoelectric plate 1223c are accommodated in the insulation frame 1224. The conducting electrode 1225b of the conductive frame 1225 is electrically connected to the piezoelectric plate 1223c. In the embodiment, the piezoelectric carrying plate 1223a and the adjusting resonance plate 1223b are made by a conductive material. The piezoelectric carrying plate 1223a includes a piezoelectric pin 1223d. The piezoelectric pin 1223d and the conducting pin 1225a are electrically connected to a driving circuit (not shown) of the driving circuit board 123, so as to receive a driving signal, such as a driving frequency and a driving voltage. Through this structure, a circuit is formed by the piezoelectric pin 1223d, the piezoelectric carrying plate 1223a, the adjusting resonance plate 1223b, the piezoelectric plate 1223c, the conducting electrode 1225b, the conductive frame 1225 and the conducting pin 1225a for transmitting the driving signal. Moreover, the insulation frame 1224 is insulated between the conductive frame 1225 and the actuator element 1223, so as to avoid the occurrence of a short circuit. Thereby, the driving signal is transmitted to the piezoelectric plate 1223c. After receiving the driving signal such as the driving frequency and the driving voltage, the piezoelectric plate 1223c deforms due to the piezoelectric effect, and the piezoelectric carrying plate 1223a and the adjusting resonance plate 1223b are further driven to generate the bending deformation in the reciprocating manner.

Furthermore, in the embodiment, the adjusting resonance plate 1223b is located between the piezoelectric plate 1223c and the piezoelectric carrying plate 1223a and served as a cushion between the piezoelectric plate 1223c and the piezoelectric carrying plate 1223a. Thereby, the vibration frequency of the piezoelectric carrying plate 1223a is adjustable. Basically, the thickness of the adjusting resonance plate 1223b is greater than the thickness of the piezoelectric carrying plate 1223a, and the vibration frequency of the actuator element 1223 can be adjusted by adjusting the thickness of the adjusting resonance plate 1223b. In the embodiment, the gas-injection plate 1221, the chamber frame 1222, the actuator element 1223, the insulation frame 1224 and the conductive frame 1225 are stacked and positioned in the gas-guiding-component loading region 1215 sequentially, so that the piezoelectric actuator 122 is supported and positioned in the gas-guiding-component loading region 1215. A plurality of clearances 1221c are defined between the suspension plate 1221a of the gas-injection plate 1221 and an inner edge of the gas-guiding-component loading region 1215 for gas flowing therethrough.

A flowing chamber 1227 is formed between the gas-injection plate 1221 and the bottom surface of the gas-guiding-component loading region 1215. The flowing chamber 1227 is in communication with the resonance chamber 1226 between the actuator element 1223, the chamber frame 1222 and the suspension plate 1221a through the hollow aperture 1221b of the gas-injection plate 1221. By controlling the vibration frequency of the gas in the resonance chamber 1226 to be close to the vibration frequency of the suspension plate 1221a, the Helmholtz resonance effect is generated between the resonance chamber 1226 and the suspension plate 1221a, so as to improve the efficiency of gas transportation. When the piezoelectric plate 1223c is moved away from the bottom surface of the gas-guiding-component loading region 1215, the suspension plate 1221a of the gas-injection plate 1221 is driven to move away from the bottom surface of the gas-guiding-component loading region 1215 by the piezoelectric plate 1223c. In that, the volume of the flowing chamber 1227 is expanded rapidly, the internal pressure of the flowing chamber 1227 is decreased to form a negative pressure, and the gas outside the piezoelectric actuator 122 is inhaled through the clearances 1221c and enters the resonance chamber 1226 through the hollow aperture 1221b. Consequently, the pressure in the resonance chamber 1226 is increased to generate a pressure gradient. When the suspension plate 1221a of the gas-injection plate 1221 is driven by the piezoelectric plate 1223c to move toward the bottom surface of the gas-guiding-component loading region 1215, the gas in the resonance chamber 1226 is discharged out rapidly through the hollow aperture 1221b, and the gas in the flowing chamber 1227 is compressed, thereby the converged gas is quickly and massively ejected out of the flowing chamber 1227 under the condition close to an ideal gas state of the Benulli's law, and transported to the ventilation hole 1215a of the gas-guiding-component loading region 1215.

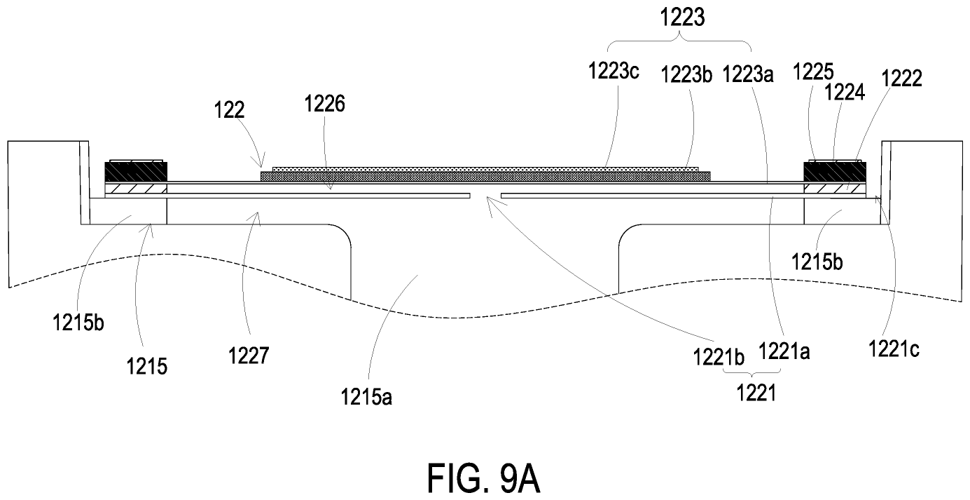

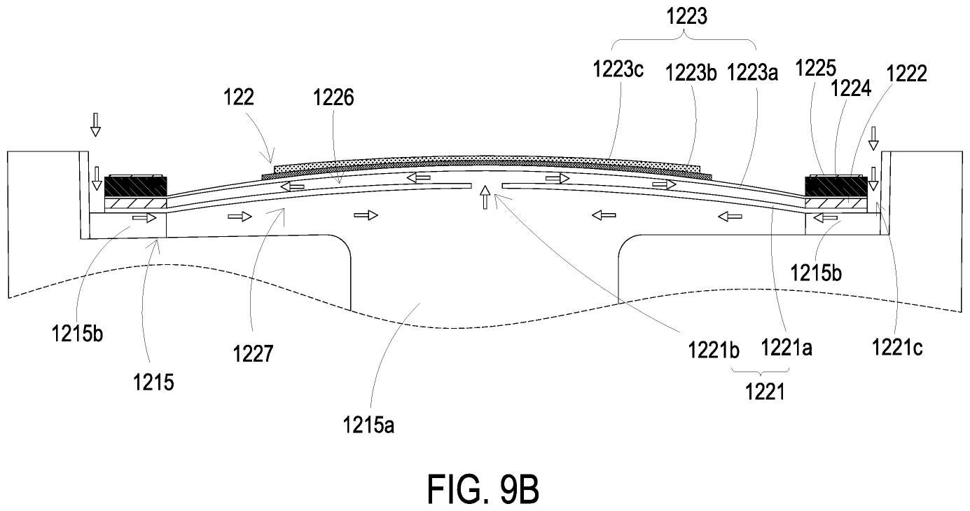

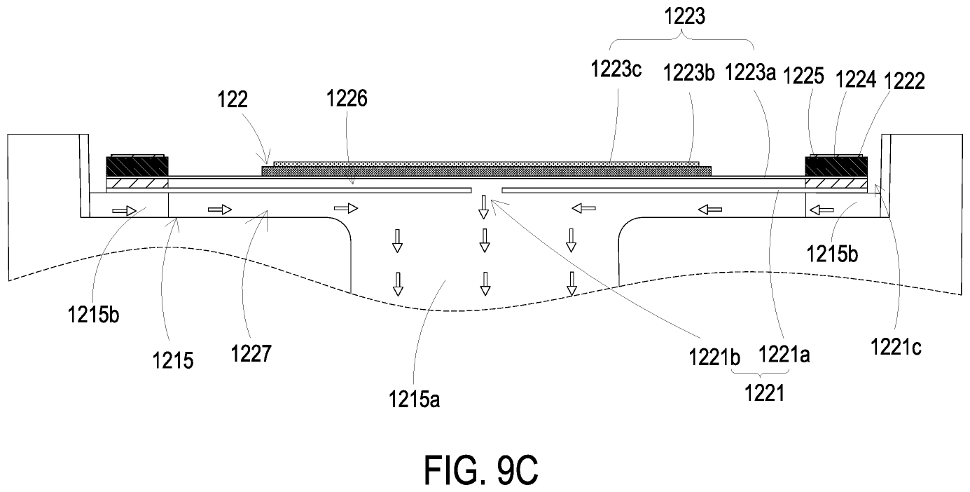

By repeating the above operation steps shown in FIG. 9B and FIG. 9C, the piezoelectric plate 1223c is driven to generate the bending deformation in a reciprocating manner. According to the principle of inertia, since the gas pressure inside the resonance chamber 1226 is lower than the equilibrium gas pressure after the converged gas is ejected out, the gas is introduced into the resonance chamber 1226 again. Moreover, the vibration frequency of the gas in the resonance chamber 1226 is controlled to be close to the vibration frequency of the piezoelectric plate 1223c, so as to generate the Helmholtz resonance effect to achieve the gas transportation at high speed and in large quantities.

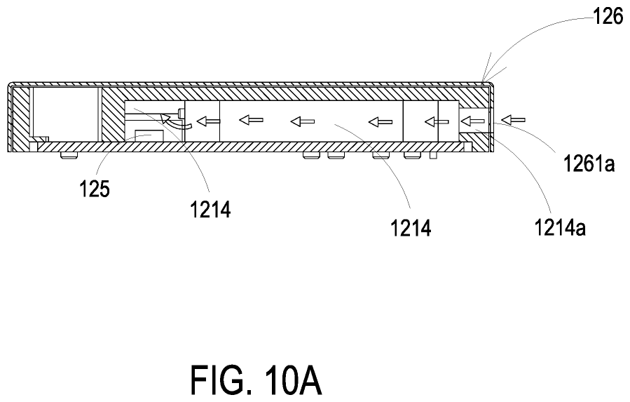

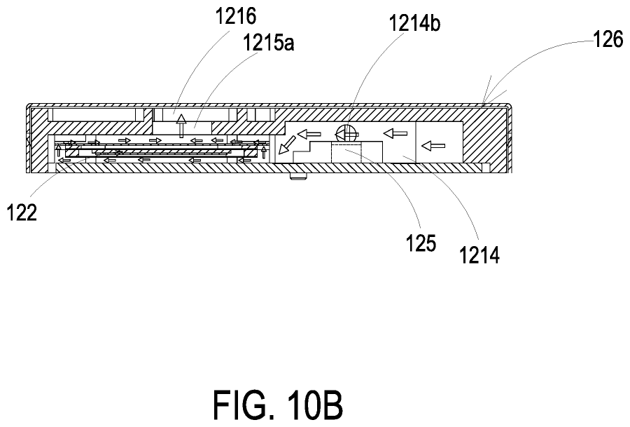

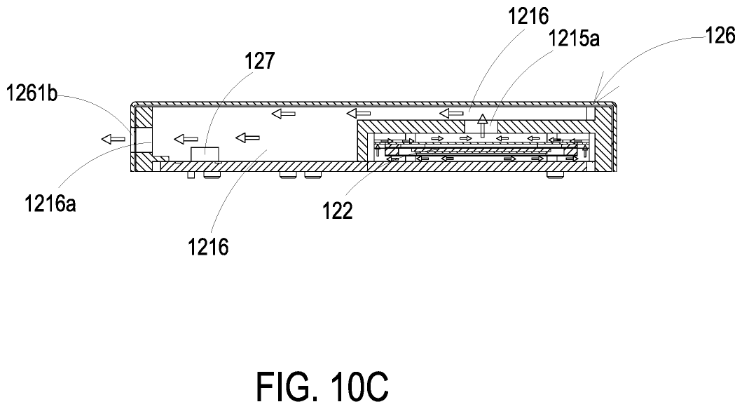

Please refer to FIG. 10A to FIG. 10C. The gas is inhaled through the gas-inlet 1214a on the outer cover 126, flows into the gas-inlet groove 1214 of the base 121 through the gas-inlet 1214a, and is transported to the position of the particulate sensor 125. The piezoelectric actuator 122 is enabled continuously to inhale the gas into the inlet path, and facilitate the gas outside the gas detection module to be introduced rapidly, flow stably, and transported above the particulate sensor 125. At this time, a projecting light beam emitted from the laser component 124 passes through the transparent window 1214b to irritate the suspended particles contained in the gas flowing above the particulate sensor 125 in the gas-inlet groove 1214. When the suspended particles contained in the gas are irradiated and generate scattered light spots, the scattered light spots are received and calculated by the particulate sensor 125 for obtaining related information about the sizes and the concentration of the suspended particles contained in the gas. Moreover, the gas above the particulate sensor 125 is continuously driven and transported by the piezoelectric actuator 122, flows into the ventilation hole 1215a of the gas-guiding-component loading region 1215, and is transported to the gas-outlet groove 1216. At last, after the gas flows into the gas outlet groove 1216, the gas is continuously transported into the gas-outlet groove 1216 by the piezoelectric actuator 122, and thus the gas in the gas-outlet groove 1216 is pushed to discharge through the gas-outlet 1216a and the outlet opening 1261b.

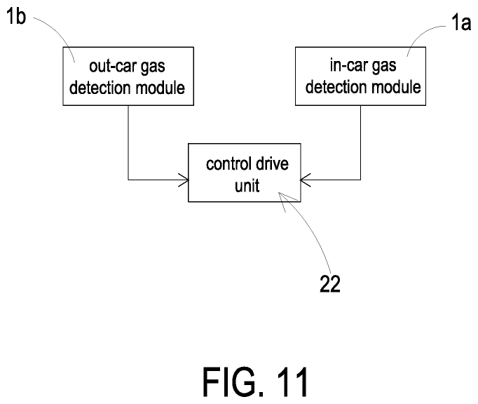

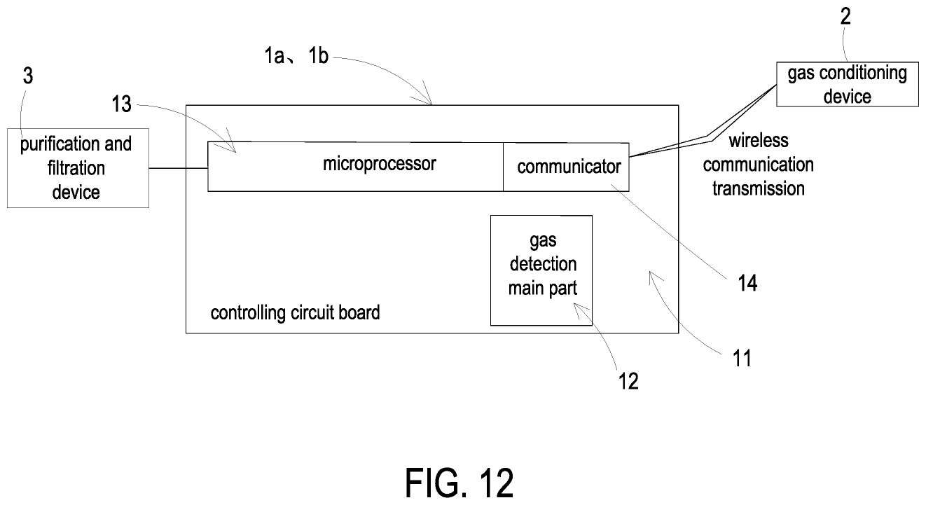

Accordingly, in view of the above description of the present disclosure, as shown in FIG. 11 and FIG. 12, the plurality of in-car gas detection modules 1a, the plurality of out-car gas detection modules 1b, the gas conditioning device 2 and the plurality of filtering and purification components D are installed in a car. The control drive unit 22 of the gas conditioning device 2 receives and compares the gas detection data of the plurality of in-car gas detection modules 1a and a plurality of out-car gas detection modules lb under the calculation of artificial intelligence, so as to locate the position of the air pollution source in the interior space A of the car. Moreover, the filtering and purification component D and the purification and filtration device 3 disposed adjacent to the location of the air pollution source are intelligently selected and controlled to be enabled to execute the filtering process, so as to provide clean, safe and breathable air in the interior space A of the car.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. An in-car air pollution prevention system for exchanging and filtering air pollution source in an interior space of a car, comprising:
a plurality of gas detection modules for detecting a status of gas and the air pollution source, and transmitting at least one gas detection datum;
a gas conditioning device for controlling the introduction or not introduction of external gas in an exterior space outside the car into the interior space of the car, wherein the gas conditioning device comprises a ventilation channel, a control drive unit and a gas guider, wherein the ventilation channel includes at least one air outlet, at least one air inlet and an external-gas inlet and the gas guider is disposed within the ventilation channel, wherein the control drive unit receives and compares the gas detection data outputted by the gas detection modules, and the gas guider guides the discharging of the gas from the at least one air outlet, and the inhaling of the gas through the external-gas inlet and the at least one air inlet; and
a plurality of filtering and purification components, wherein at least one of the plurality of filtering and purification components is disposed at the at least one air outlet for filtering and purifying the external gas and the air pollution source, and at least one of the plurality of filtering and purification components is disposed at the at least one air inlet for filtering and purifying the air pollution source;
wherein after the control drive unit compares the gas detection data, the gas conditioning device is allowed to intelligently select and control the introduction or not introduction of the external gas in the exterior space outside the car into the interior space of the car, and the gas guider of the gas conditioning device is instantly controlled to be enable by the plurality of gas detection modules in a state of a monitoring mechanism for filtering and purifying the air pollution source in the interior space of the car through the plurality of filtering and purification components, so that the air pollution source in the interior space of the car is filtered and exchanged to provide clean air.

2. The in-car air pollution prevention system according to claim 1, wherein the air pollution source is at least one selected from the group consisting of suspended particles, carbon monoxide, carbon dioxide, ozone, sulfur dioxide, nitrogen dioxide, lead, total volatile organic compounds (TVOC), formaldehyde, bacteria, fungi, virus and a combination thereof.

3. The in-car air pollution prevention system according to claim 1, wherein the filtering and purification component comprises an activated carbon and a high efficiency particulate air (HEPA) filter screen.

4. The in-car air pollution prevention system according to claim 1, wherein the state of the monitoring mechanism is enabled when at least one of the gas detection data of the air pollution source detected by the plurality of gas detection modules in the interior space of the car is greater than a safe detection value.

5. The in-car air pollution prevention system according to claim 4, wherein the safe detection value is at least one selected from the group consisting of PM2.5 less than 35 μg/m³, carbon dioxide content less than 1000 ppm, total volatile organic compounds (TVOC) less than 0.56 ppm, formaldehyde content less than 0.08 ppm, the amount of bacteria less than 1500 CFU/m³, the amount of fungi less than 1000 CFU/m³, sulfur dioxide content less than 0.075 ppm, nitrogen dioxide content less than 0.1 ppm, carbon monoxide content less than 9 ppm, ozone content less than 0.06 ppm, lead content less than 0.15 μg/m³ and a combination thereof.

6. The in-car air pollution prevention system according to claim 1, wherein each of the plurality of gas detection module comprises a controlling circuit board, a gas detection main part, a microprocessor and a communicator, and the gas detection main part, the microprocessor and the communicator are integrally packaged on the controlling circuit board and electrically connected to the controlling circuit board, wherein the microprocessor controls the detection of the gas detection main part, the gas detection main part detects the air pollution source and outputs a detection signal, the microprocessor receives the detection signal for calculating, processing and outputting, so that the respective microprocessor of the respective gas detection module generates the gas detection datum, so as to provide to the respective communicator for external communication transmission.

7. The in-car air pollution prevention system according to claim 6, wherein the gas detection main part comprises:
a base comprising:
a first surface;
a second surface opposite to the first surface;
a laser loading region hollowed out from the first surface to the second surface;
a gas-inlet groove concavely formed from the second surface and disposed adjacent to the laser loading region, wherein the gas-inlet groove comprises a gas-inlet and two lateral walls, and two transparent windows are opened on the two lateral walls and is in communication with the laser loading region;
a gas-guiding-component loading region concavely formed from the second surface and in communication with the gas-inlet groove, wherein a ventilation hole penetrates a bottom surface of the gas-guiding-component loading region, and
a gas-outlet groove concavely formed from the first surface in a region spatially corresponding to the bottom surface of the gas-guiding-component loading region, and hollowed out from the first surface to the second surface in a region where the first surface is not aligned with the gas-guiding-component loading region, wherein the gas-outlet groove is in communication with the ventilation hole, and a gas-outlet is disposed in the gas-outlet groove and in communication with the environment outside the base;
a piezoelectric actuator accommodated in the gas-guiding-component loading region;
a driving circuit board covering and attached to the second surface of the base;
a laser component positioned and disposed on the driving circuit board, electrically connected to the driving circuit board, and accommodated in the laser loading region, wherein a light beam path emitted from the laser component passes through the transparent window and extends in a direction perpendicular to the gas-inlet groove;
a particulate sensor positioned and disposed on the driving circuit board, electrically connected to the driving circuit board, and disposed at a position where the gas-inlet groove orthogonally intersects with the light beam path of the laser component, so that suspended particles contained in the air pollution source passing through the gas-inlet groove and irradiated by a projecting light beam emitted from the laser component are detected;
a gas sensor positioned and disposed on the driving circuit board, electrically connected to the driving circuit board, and accommodated in the gas-outlet groove, so as to detect the air pollution source introduced into the gas-outlet groove; and
an outer cover covering the base and comprising a side plate, wherein the side plate has an inlet opening and an outlet opening, the inlet opening is spatially corresponding to the gas-inlet of the base, and the outlet opening is spatially corresponding to the gas-outlet of the base;
wherein the outer cover covers the first surface of the base, and the driving circuit board covers the second surface of the base, so that an inlet path is defined by the gas-inlet groove, and an outlet path is defined by the gas-outlet groove, thereby the air pollution source outside the gas-inlet of the base is inhaled by the piezoelectric actuator, transported into the inlet path defined by the gas-inlet groove through the inlet opening, and passes through the particulate sensor to detect the particle concentration of the suspended particles contained in the air pollution, and then the air pollution source transported through the piezoelectric actuator is transported out of the outlet path defined by the gas-outlet groove through the ventilation hole, passes through the gas sensor for detecting, and discharged through the outlet opening.

8. The in-car air pollution prevention system according to claim 7, wherein the particulate sensor detects suspended particulate information.

9. The in-car air pollution prevention system according to claim 7, wherein the gas sensor comprises a volatile-organic-compound sensor detecting carbon dioxide or volatile organic compounds information.

10. The in-car air pollution prevention system according to claim 7, wherein the gas sensor comprises at least one selected from the group consisting of a formaldehyde sensor, a bacteria sensor, a virus sensor and a combination thereof, wherein the formaldehyde sensor detects formaldehyde gas information, the bacteria sensor detects bacteria or fungi information, and the virus sensor detects virus gas information.

11. The in-car air pollution prevention system according to claim 1, wherein the plurality of gas detection modules comprises at least one out-car gas detection module and at least one in-car gas detection module, the at least one out-car gas detection module is disposed in the external-gas inlet for detecting the external gas in the exterior space outside the car and transmitting an out-car gas detection datum, and the at least one in-car gas detection module is disposed in the interior space of the car for detecting the air pollution source in the interior space of the car and transmitting at least one in-car gas detection datum.

12. The in-car air pollution prevention system according to claim 11, wherein the gas conditioning device further comprises an intake-control element, the external-gas inlet is used to introduce the external gas in the exterior space outside the car, and the air inlet is used to introduce the air pollution source in the interior space of the car, wherein the intake-control element is controlled by the control drive unit to select the opening of the external-gas inlet, and the control drive unit receives and compares the out-car gas detection datum outputted by the out-car gas detection module and the in-car gas detection datum output by the in-car gas detection module to determine if the external-gas inlet need to be opened, so as to control the introduction or non-introduction of the external gas in the exterior space outside the car or the inhalation of the air pollution source in the interior space of the car through the air inlet.

13. The in-car air pollution prevention system according to claim 11, wherein the control drive unit receives and compares the in-car gas detection data outputted by at least three in-car gas detection modules under the calculation of artificial intelligence, so as to locate the position of the air pollution source in the interior space of the car, and intelligently select and control the filtering and purification component disposed in the position of the air inlet adjacent to the air pollution source to accelerate the transportation for filtering.

14. The in-car air pollution prevention system according to claim 11, wherein the control drive unit receives and compares the in-car gas detection data outputted by at least three in-car gas detection modules under the calculation of artificial intelligence, so as to locate the position of the air pollution source in the interior space of the car, and intelligently select and control the air outlet adjacent to the air pollution source to expel gas in first priority, and direct the air pollution source toward the air inlet adjacent thereto, wherein the control drive unit of the gas conditioning device selects and controls the rest of the air outlets to expel gas under the calculation of artificial intelligence, so that an airflow is generated to direct the air pollution source toward the air inlet adjacent to the air pollution source to be inhaled for filtering rapidly.

15. The in-car air pollution prevention system according to claim 11, further comprising at least one purification and filtration device, and the purification and filtration device includes the gas guider and the filtering and purification component, and the in-car gas detection module is in combination with the purification and filtration device to control the enablement of the gas guider, so that the air pollution source in the interior space of the car is guided to enter the filtering and purification component of the purification and filtration device for filtering and purifying.

16. The in-car air pollution prevention system according to claim 15, wherein the control drive unit of the gas conditioning device receives and compares the in-car gas detection data outputted by at least three in-car gas detection modules under the calculation of artificial intelligence, so as to locate the position of the air pollution source in the interior space of the car, and intelligently select and control the enablement of the purification and filtration device adjacent to the air pollution source, so that the air pollution source can be inhaled into the purification and filtration device without diffusion and accelerate the filtration.

17. The in-car air pollution prevention system according to claim 15, wherein the control drive unit of the gas conditioning device receives and compares the in-car gas detection data outputted by at least three in-car gas detection modules under the calculation of artificial intelligence, so as to locate the position of the air pollution source in the interior space of the car, and intelligently select and control the purification and filtration device adjacent to the air pollution source to be enabled in first priority, wherein the control drive unit of the gas conditioning device selects and controls the rest of the purification and filtration devices to be enabled under the calculation of artificial intelligence, so that an airflow is generated to direct the air pollution source toward the purification and filtration device adjacent to the air pollution source to be inhaled for filtering rapidly.

18. The in-car air pollution prevention system according to claim 15, wherein the at least one purification and filtration device is in combination with and embedded on a trim panel, a seat or a door pillar in the interior space of the car, and the in-car gas detection module transmits the in-car gas detection datum for being received and compared by the control drive unit of the gas conditioning device under the calculation of artificial intelligence, and intelligently selecting and controlling the enablement of the purification and filtration device adjacent to the air pollution source.

19. The in-car air pollution prevention system according to claim 15, wherein the in-car gas detection module is in combination with a wearable device worn on a human body to detect the air pollution source in the interior space of the car in real-time, and transmit the in-car gas detection datum in the interior space of the car for receiving and comparing by the control drive unit of the gas conditioning device under the calculation of artificial intelligence, and intelligently selecting and controlling the enablement of the purification and filtration device adjacent to the air pollution source.

20. The in-car air pollution prevention system according to claim 15, wherein at least one of the plurality of in-car gas detection modules is disposed at two sides of the plurality of purification and filtration devices, so that the control drive unit receives and compares the in-car gas detection data outputted by the in-car gas detection modules at the positions of the plurality of the purification and filtration devices, so as to make sure that the air pollution source is filtered by the plurality of the purification and filtration devices to generate clean air introduced into the interior space of the car.

21. The in-car air pollution prevention system according to claim 3, wherein the service life of the HEPA filter screen is calculated based on the monitoring mechanism of the gas detection data detected by the in-car gas detection module and the out-car gas detection module with reference to an accumulated operation time of the gas guider in the gas conditioning device.

22. The in-car air pollution prevention system according to claim 1, wherein the control drive unit comprises a touch screen for setting up the control instructions of the gas conditioning device by touching the touch screen and displaying the gas detection datum.
